(19) United States
(12) Patent Application Publication
(10) Pub. No.: US 2009/0143869 A1
(43) Pub. Date: Jun. 4, 2009

(54) ANTI-SHOCK ARTIFICIAL KNEE JOINT STRUCTURE

(76) Inventors: CHIA-PAO CHENG, Shu Lin City (TW); Fu-Kuo Wu, Panchiao City (TW)

Correspondence Address: LEONG C LEI, PMB # 1008, 1867 YGNACIO VALLEY ROAD, WALNUT CREEK, CA, 94598, US

(21) Appl. No.: 11/946,880

(22) Filed: Nov. 29, 2007

(51) Int. Cl. A61F 2/62

(52) U.S. Cl. 623/39

(57) ABSTRACT

An anti-shock artificial knee joint includes a pivot stop block forming a first pivot hole rotatably receiving therein a first pivot for bending the knee joint and a second pivot hole rotatably receiving a second pivot. A gap extending from the first pivot hole so that when the pivot stop block is subjected to an external force induced in the process of walking, the gap is reduced to secure the first pivot from rotation whereby the pivot stop block rotates about the second pivot to cause the pivot stop block to engage spring-biased pistons to thereby absorb the external force or the impact caused by the external force. The spring-biased pistons are provided with set screws to adjust the cushioning effect provided by the spring-biased pistons.

### BACKGROUND OF THE INVENTION

(a) Technical Field of the Invention

The present invention relates to an anti-shock artificial knee joint structure, which uses linking members and pivots to form at least two movable jointing portions, which together with a pivot stop block and a pneumatic cylinder assembly, allows the knee joint to operate in a safe and smooth manner and constituting an anti-shock device that improves the impact acting upon the body of a disabled person walking with an artificial limb to thereby ensure a comfortable and smooth operation of the knee joint.

(b) Description of the Prior Art

With the development and progress in medical technology, disabled persons whose legs are damaged and not allowed to walk naturally can now be rehabilitated with the waling capability by installing an artificial limb that incorporates with an artificial knee joint. The artificial knee joint of the artificial limb is a mechanical device that is designed to simulate the operation of a natural knee joint in order to provide the most natural waking capability to the disabled persons.

Since the natural nee joint is of a very complicated structure, although the conventional artificial knee joint employs a mechanical structure that is effectively to substantially simulate the operation of the natural knee joint, the conventional knee joint, in consideration of structural strength and manufacturing costs, cannot exactly duplicate the operation of the natural knee joint and thus is not able to provide a user with the capability of walking in a natural way. For an adult to take a walk with natural legs, a leg is standing upright to support the adult's body weight, and another leg is raised and then put forward and lowered down to set on the ground. Before it gets contact with the ground, the knee joints of both legs are slightly bent or curved in order to coordinate with the foot contacting the ground to alleviate the impact acting upon the adult's body by the reaction force from the ground at the moment that the foot contacts the ground and to set the posture for the next step. This is the ideal way of waling. However, for a disabled person walking with an artificial leg, when the artificial leg is raised and put forward by the force of the thigh, the portion of the artificial leg that corresponds to a lower leg or shank of a natural leg is not subject to any control over the posture of the foot contacting the ground and the angle that the knee joint bends. Thus, when the foot of the artificial leg gets contact with the ground, most of the weight of the disabled person falls on the heel portion of the artificial leg and an impact caused by the reaction force is transmitted through the spine of the disabled person to the brain and other organs of the person. When waling in his way for a long time, the shock may cause potential damages or discomfort to the body. Clearly enough, the conventional artificial knee joint cannot satisfy the need of the modern users, and improvement is thus required.

### SUMMARY OF THE INVENTION

The purpose of the present invention is to provide an anti-shock artificial knee joint, comprising an upper-section linking member forming a connection head for coupling with a thigh and a lower-section pneumatic cylinder housing having a lower end coupled to a shank. An intermediate-section pivot extends through upper trough holes of an intermediate-section linking member and a trough pivot hole of a pivot stop block. The pivot stop block has an end coupled to upper-section-linking-member through holes defined in the upper-section linking member. The lower-section pneumatic cylinder housing has an upper end coupled to lower through holes of an intermediate-section linking member and the lower-section pneumatic cylinder housing contains therein a pneumatic cylinder assembly having a pneumatic cylinder link rod connected to the pivot stop block. With the coordination of the above components with the pneumatic cylinder assembly and the pneumatic cylinder link rod, the knee joint is allowed to bend and return to the unbent position. Further, the lower-section pneumatic cylinder housing forms two sloped bores in which pistons operating with springs and elastic bodies are movably received for provide cushion effect and set screws being provided to adjust cushioning effect, which together constitute an anti-shock device.

When walking with the knee joint, at the time that the limb gets contact with the ground, the weigh of a user's body falls on the knee joint, causing the pivot stop block to tightly clamp the pivot thereby preventing undesired bending of the knee joint and also transmitting the weight trough the intermediate-section linking member to the anti-shock device to alleviate the impact caused by contact with the ground, allowing a user of the knee joint to walk in a more natural and comfortable manner.

The foregoing object and summary provide only a brief introduction to the present invention. To fully appreciate these and other objects of the present invention as well as the invention itself all of which will become apparent to those skilled in the art, the following detailed description of the invention and the claims should be read in conjunction with the accompanying drawings. Throughout the specification and drawings identical reference numerals refer to identical or similar parts.

Many other advantages and features of the present invention will become manifest to those versed in the art upon making reference to the detailed description and the accompanying sheets of drawings in which a preferred structural embodiment incorporating the principles of the present invention is shown by way of illustrative example.

### BRIEF DESCRIPTION OF THE DRAWINGS

FIG. 1 is an exploded view showing an anti-shock artificial knee joint constructed in accordance with the present invention;

FIG. 2 is a cross-sectional view of the anti-shock artificial knee joint of the present invention;

FIG. 3 is a cross-sectional view of the anti-shock artificial knee joint of the present invention in a different position;

FIG. 4 is a cross-sectional view of the anti-shock artificial knee joint of the present invention in a finer different position;

FIG. 5 shows a sequence of drawings illustrating the operation of the knee joint of the present invention; and

FIG. 6 shows drawings illustrating cushioning operation of the knee joint of the present invention.

### DETAILED DESCRIPTION OF THE PREFERRED EMBODIMENTS

The following descriptions are of exemplary embodiments only, and are not intended to limit the scope, applicability or configuration of the invention in any way. Rather, the following description provides a convenient illustration for implementing exemplary embodiments of the invention Various changes to the described embodiments may be made in the function and arrangement of the elements described without departing from the scope of the invention as set forth in the appended claims.

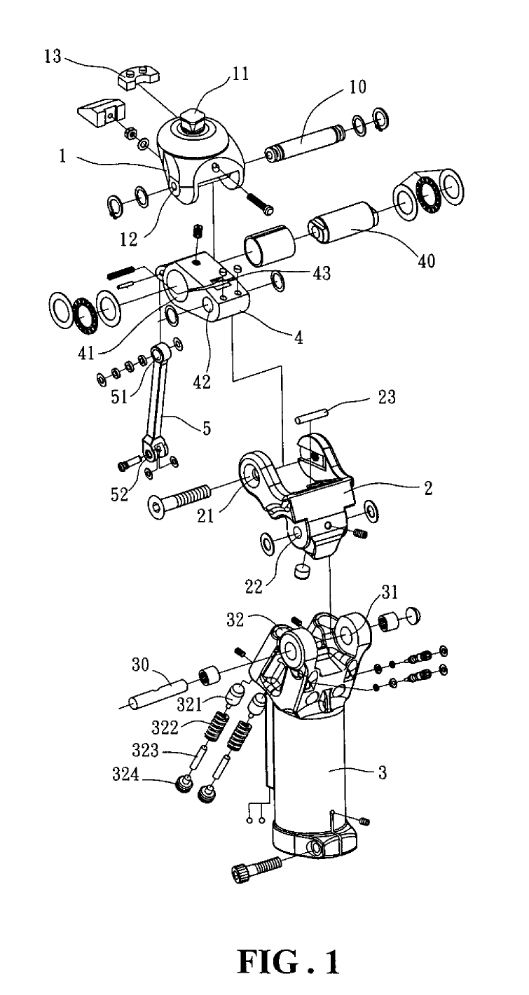

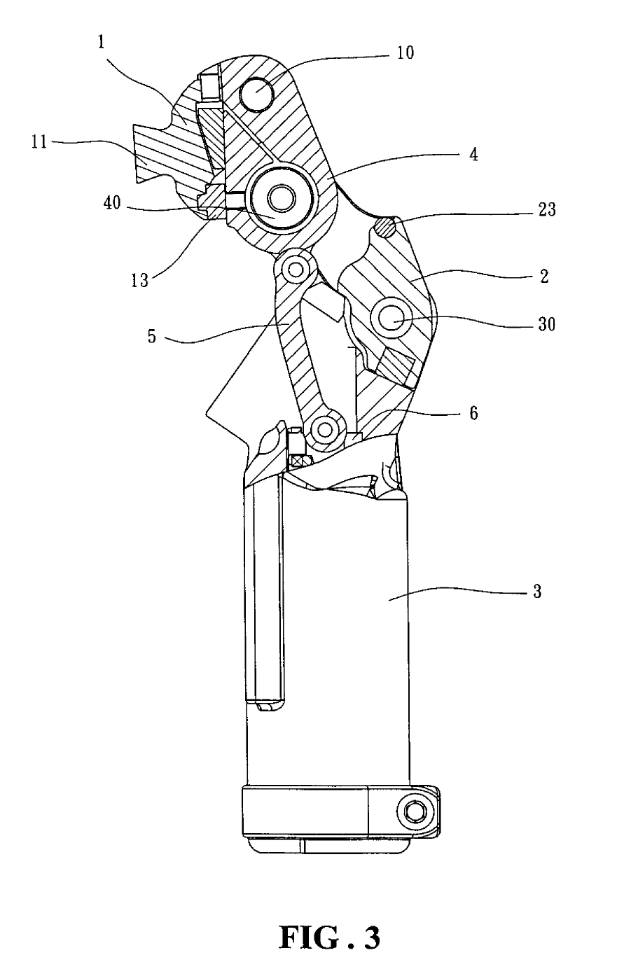

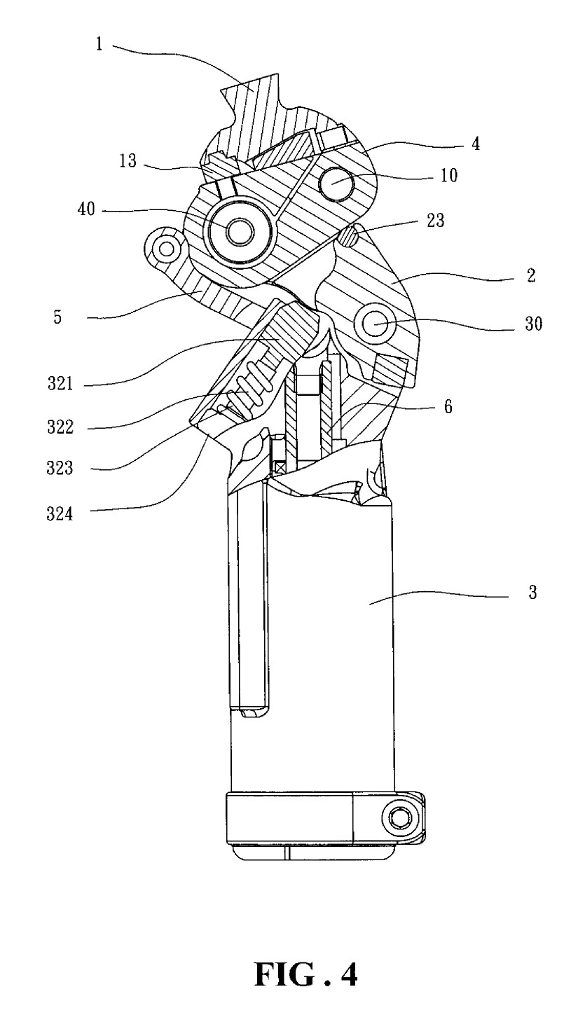

The present invention provides an anti-shock artificial knee joint of which the structure and operation will be now described in detail with reference to the attached drawings for a full understanding of the present invention. As shown in FIGS. 1-6, the anti-shock artificial knee joint in accordance with the present invention has a structure that is comprised of an upper-section linking member 1, an intermediate-section linking member 2, a lower-section pneumatic cylinder housing 3, a pivot stop block 4, a pneumatic cylinder link rod 5, and a pneumatic cylinder assembly 6.

As shown in FIG. 1 of the attached drawings, the upper-section linking member 1 has an upper end to which a connection head 11 is mounted. A thigh of a user (not shown) of the artificial knee joint, which can be for example a portion of an artificial limb or a natural thing with suitable coupling means, is coupled to the connection head 11 by means of bolts (not shown). An upper-section pivot 10 extends trough a through hole 42 defined in the pivot stop block 4 and also extends upper-section-lining-member through holes 12 defined in the upper-section linking member 1. An intermediate-section pivot 40 extends through upper through holes 21 of the intermediate-section lining member 2 and a pivot hole 41 formed through the pivot stop block 4. A lower-section pivot 30 extends through lower-section-pivot holes 31 defined in the lower-section pneumatic cylinder housing 3 and lower through holes of the intermediate-section linking member 2. The pneumatic cylinder link rod 5 has a lower end 52 coupled to the pneumatic cylinder assembly 6 received in the lower-section pneumatic cylinder housing 3 and an upper end 51 coupled to the pivot stop block 4.

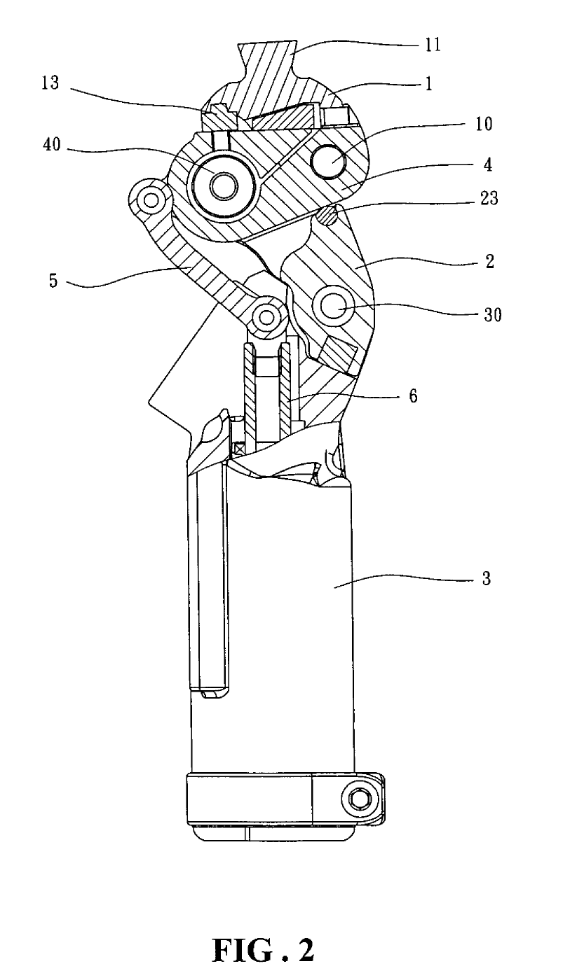

Referring to FIGS. 2-4, the upper-section lining member 1 and the pivot stop block 4 are rotatable about the intermediate-section pivot 40 to perform a knee bending operation, which is carried out in smooth manner with the aid of the pneumatic-cylinder link rod 5 bat is coupled to the pivot stop block 4 and the pneumatic cylinder assembly 6. When the knee returns back the original unbent posture, a cushion seat 13 mounted to and between the upper-section linking member 1 and the pivot stop block 4 and a cushion block mounted to the intermediate-section linking member 2 eliminate the impact and/or shock caused by the returning movement of the above components so as to ensure smooth bending and returning operations of the anti-shock artificial knee joint of the preset invention.

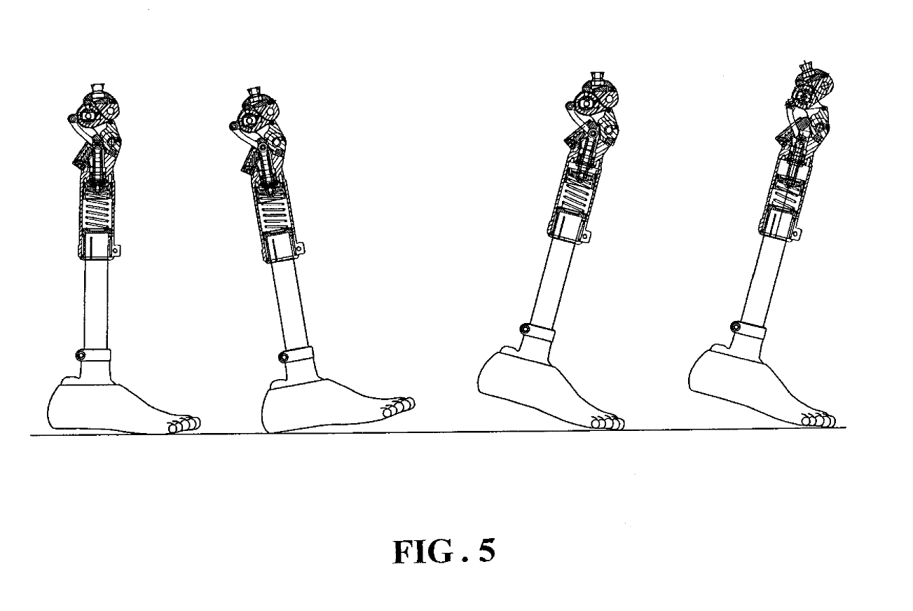

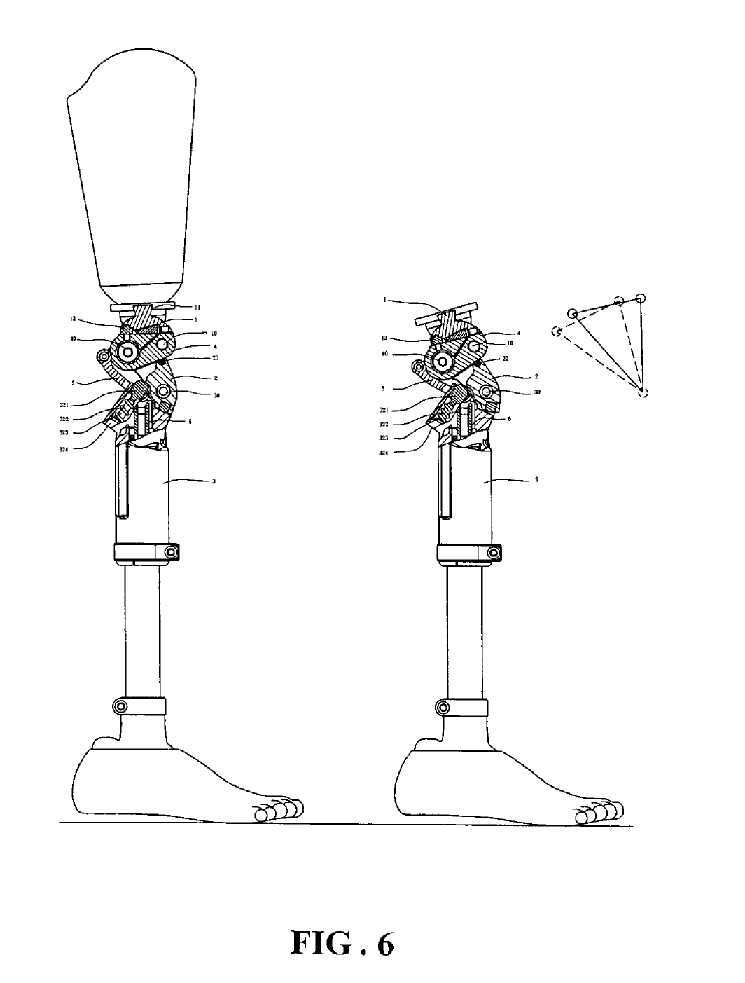

When a disabled person attempts to walk with an artificial limb, since the artificial knee cannot operate in the same way as natural knee joints to take a slight bending for realizing cushion effect and getting ready for the next step, a reaction force generated at the instance when the artificial limb gets contact with the ground causes a great impact or shock, which makes the disabled person wearing the artificial limb uncomfortable. To cope such a problem, in accordance with the present invention, two sloped through holes 32 are defined in the lower-section pneumatic cylinder housing 3 and each sloped bore 32 contains therein a piston 321, a spring 322, an elastic body 323, and a set screw 324, which are arranged to engage and thus support the intermediate-section linking member 2 to thereby form, together with the pivot stop block 4, an anti-shock device. As shown in FIGS. 5 and 6, to walk with an artificial limb incorporating the artificial knee joint of the present invention, one leg of the artificial limb user stands upright for bearing the gravity of the user's body, while the other leg is raised in a slightly bent manner. Said the other leg is moved forward and then lowered down to step on the ground, at which moment the gravity or weight of the user's body is moved forward to set on the anti-shock knee joint of the present invention. Due to a gap 43 formed in the pivot stop block 4 and jointing the pivot hole 41, when the user's body weight falls onto the pivot stop block 4, the gap 43 is reduced, depressing and thus securing the intermediate-section pivot 40 from rotation with respect to the pivot hole 41. The lower through holes 22 of the intermediate-section liking member 2 are thus rotated about the lower-section pivot 30, causing the anti-shock device that engages and supports the intermediate-section linking member 2 to be compressed and thus absorbing the impact or shock and the reaction force caused by contact with the ground. In addition, the set screw 324 provided at a lower portion of each sloped bore 32 ensures adjustment of cushioning effect of the anti-shock device to make walking with the artificial knee joint more comfortable and smoother.

It will be understood that each of the elements described above, or two or more together may also find a useful application in other types of methods differing from the type described above.

While certain novel features of this invention have been shown and described and are pointed out in the annexed claim, it is not intended to be limited to the details above, since it will be understood that various omissions, modifications, substitutions and changes in the forms and details of the device illustrated and in its operation can be made by those skilled in the art without departing in any way from the spirit of the present invention.

I claim:

1. An anti-shock knee joint, comprising:
an upper-section linking member having an upper end forming a connection head adapted to couple to a thigh of a limb and a lower end defining upper-section-linking-member through holes;
a pivot stop block forming a through pivot hole and a through hole, the pivot stop block defining a gap jointing the trough pivot hole;
a cushion seat arranged between the upper-section linking member and the pivot stop block;
an intermediate-section linking member comprising a cushion block and forming upper and lower through holes;
a lower-section pneumatic cylinder housing forming lower-section-pivot holes and sloped bores, each sloped bore containing therein a spring and an elastic body and further containing a piston in an upper portion thereof and a set screw in a lower portion thereof;
an upper-section pivot extending through the through hole of the pivot stop block and the upper-section-linking member through holes defined in the upper-section linking member;
an intermediate-section pivot extending through the upper through holes of the intermediate-section linking member and the through pivot hole of the pivot stop block;
a lower-section pivot extending the lower-section-pivot holes of the lower-section pneumatic cylinder housing and the lower rough holes of the intermediate-section linking member,
a pneumatic cylinder assembly arranged inside the lower-section pneumatic cylinder housing; and
a pneumatic cylinder link rod having an upper end coupled to the pivot stop block and a lower end coupled to the pneumatic cylinder assembly;
wherein when the pivot stop block is subjected to an external force applied thereto, the gap is reduced to tightly secure the intermediate-section pivot from rotation, whereby the intermediate-section lining member is rotatable about the lower-section pivot to have the intermediate-section linking member engaging and supported by the pistons contained in the sloped bores of the lower-section pneumatic cylinder housing for employing the springs, together with the elastic bodies, to cushion a reaction force when subjected to an external force.
